(19) United States
(12) Patent Application Publication
(10) Pub. No.: US 2018/0110644 A1
(43) Pub. Date: Apr. 26, 2018

(54) COUPLING ELEMENTS FOR COUPLING STRAPS, IN PARTICULAR FOR STRAP OF BANDAGES AND ORTHOSES

(71) Applicant: BAUERFEIND AG, Zeulenroda-Triebes (DE)

(72) Inventor: Gerald STIER, Langenwetzendorf (DE)

(73) Assignee: BAUERFEIND AG, Zeulenroda-Triebes (DE)

(21) Appl. No.: 15/569,855

(22) PCT Filed: May 11, 2016
(86) PCT No.: PCT/EP2016/060589

§ 371 Date: Oct. 27, 2017

(30) Foreign Application Priority Data
May 13, 2015 (DE) 10 2015 006 329.4

(51) Int. Cl. A61F 5/02; A44B 11/00; A44B 11/22

(52) U.S. Cl. CPC A61F 5/028; A44B 11/22; A44B 11/006

(57) ABSTRACT

The present invention relates to coupling elements to couple belts, particularly abdominal belts and tension bands. The coupling elements can particularly be used for belts of braces or orthoses.

The present invention relates to coupling elements to couple belts, particularly abdominal belts and tension bands. The coupling elements can particularly be used for belts of braces or orthoses.

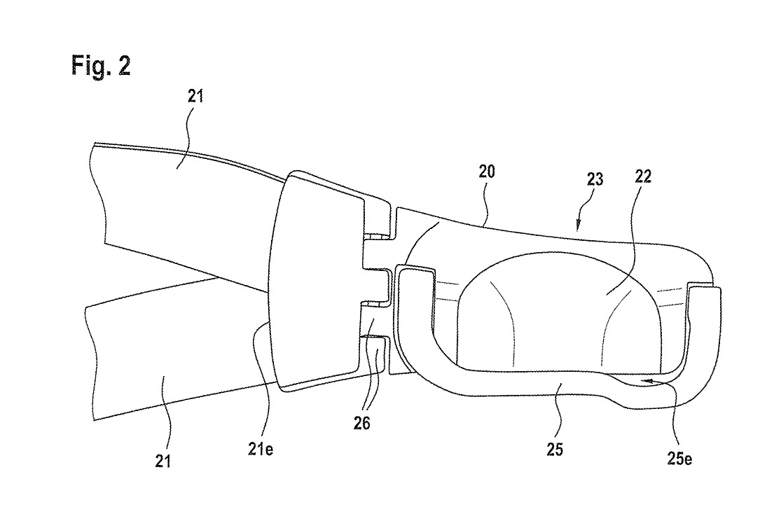

Coupling elements used to couple belts are well known, including, for example, belt buckles. Coupling elements are also used for braces and orthoses, in particular for closing abdominal belts and tension bands in the abdominal area. DE 10 2012 009 214 A1 shows, in FIG. 2, a first coupling means of a coupling element which can be coupled to a second coupling means (which is not shown), to tension the belts, which are designed as pulleys. The DE 10 2007 062 274 A1 discloses an orthopedic support device, with belts coupled to each other by means of quick release buckles.

Medical braces and orthoses are mostly used by people whose movement is restricted by an illness or injury, or because of their age, or because they are not very strong. These individuals experience difficulties using the coupling elements of the prior art when putting on the orthosis, particularly in cases where the coupling elements serve the purpose of coupling tension bands—particularly tension belts—where a certain tensile force has to be applied to couple the coupling means.

The technical problem addressed by the present invention is that of providing improved coupling elements, in particular coupling elements that are easy to couple. The technical problem addressed by the present invention is also particularly that of providing devices which make it possible to easily couple tension bands together, for example in the abdominal area, wherein the devices should exert a tensile force—specifically in the case of braces and orthoses—such that this tensile force is created.

The present invention addresses the problem by a coupling element according to claim 1.

The present invention addresses the technical problem by providing a coupling element to couple a first belt element to a second belt element, wherein the first belt element has a first coupling means on one of its ends, and wherein the second belt element has a second coupling means on one of its ends, wherein the first coupling means has a first grip element and an eyelet element with at least one eyelet, and wherein the second coupling means has a second grip element and a hook element with a hook to hook into the eyelet of the eyelet element, wherein the first grip element and/or the second grip element has a handle with a grip opening for the holding of the first and/or second grip element.

In the context of the present invention, “coupling” means the reversible joining of the two coupling means.

According to the invention, the two coupling means of the coupling element are also configured with grip elements, wherein at least one of the grip elements has a handle with a grip opening for the holding of the grip element. Using the grip with the grip opening, the person can simply and firmly grip the coupling means and thus make the connection to the other coupling means. Any slippage can be prevented in this way. The same is true when the coupling means are released from each other.

In a preferred embodiment, the first grip element and the second grip element each have a handle with a grip opening, for holding.

In a preferred embodiment, the first grip element is positioned between the first belt element and the eyelet element.

In a preferred embodiment, the second grip element is positioned at one end of the second belt element, and the hook element with the hook is positioned on the back side of the grip element. The hook element can also be formed only by the hook.

In the context of the present invention, the ‘back side’ of the coupling element and/or of the grip element means the side which faces the body when worn. Accordingly, in the context of the present invention, the ‘front side’ of the coupling element and/or of the grip element means the side which faces away from the body when worn.

In a preferred embodiment, the first belt element and the second belt element form an abdominal belt. Particularly in the abdominal area, the grip elements with handles are advantageous because a tensile force can be applied in this case particularly well by the handle when the coupling means are coupled together or uncoupled from each other.

In a preferred embodiment, the first belt element and the second belt element form a tension band, in particular a tension belt. Accordingly, the coupling element according to the invention is preferably included for the purpose of coupling belt elements together, which then form a tension band, in particular a tension belt. When such belt elements are coupled together, a particularly great amount of force is required because the coupling means must be pulled to couple the hook into the eyelet against the tensile force of the belt elements. The handles of the grip elements in this case enable pulling the coupling elements with sufficient force, but prevent the coupling elements from slipping out of the hand.

Particularly preferably, the coupling element serves the purpose of coupling two belt elements which form a tension band, in particular a tension belt, in the abdominal area, as may be the case with braces or orthoses, for example. For the reasons given above, people with restricted movement or minimal strength are able to pull a tension band tight, in spite of the tensile force, by means of the coupling element according to the invention. This is advantageous in braces and orthoses, by way of example, which have such a tension belt, such that the brace or orthosis can be pulled tight by the person wearing the brace or orthosis as independently as possible without assistance.

Orthoses in particular, such as lumbar orthoses, frequently have tensioning devices in the form of tension band systems, wherein the tension/pressure of the applied orthosis on the body part can be increased in a controlled manner by means of these tensioning devices. Such a tensioning device is disclosed in DE 10 2012 009 214 A1, by way of example. When applied, such a tensioning device is wrapped, together with the orthosis, around the torso of the person, and the two ends of the tensioning device are connected to each other by a force fit resulting from the tensioning device being closed like a belt around the torso. A coupling element closes the device. Because, for the efficacy of such orthoses, the tensioning device must exert a relatively high tensile force, which must be opposed by an accordingly high force when the coupling means are coupled together when the orthosis is put on, the coupling elements according to the invention are particularly advantageous. The same is true for the uncoupling of the coupling means.

According to the invention, the eyelet element has at least one eyelet. Preferably, the eyelet element has a plurality of eyelets—for example, four to ten eyelets—such that the hook of the second coupling means can be hooked, according to the body size of the person, in a corresponding eyelet.

In a preferred embodiment, the eyelet element is designed in the manner of a ladder.

If the eyelet element has a plurality of eyelets, they are accordingly preferably designed in such a manner that they are formed by a ladder-like eyelet element, wherein said eyelets are the rung spaces in the ladder. The ladder-like form of the eyelet element has the advantage that the eyelet element can be curved, such that it is adapted to the basic anatomical structure of the abdomen on which the eyelet element lies.

In this case, the eyelet element, and in particular the ladder-shaped eyelet element, is preferably positioned at the outer end of the grip element and runs at least approximately horizontal in the case of an abdominal belt.

In the context of the present invention, ‘approximately horizontal’ and/or ‘horizontal’ are used to mean that an element—for example, the eyelet element—has a handle, when the belt elements which are connected to form an abdominal belt for the coupling element are worn, have an approximately-horizontal and/or horizontal course, since an abdominal belt also runs approximately horizontally when worn. The eyelet element in this case is preferably to the side of the grip element, in particular at the lateral end of the grip element.

In a preferred embodiment, the eyelet element is designed in the manner of a ladder, and is curved upwards as seen from the associated first grip element. In this embodiment, the eyelet element is oriented approximately horizontally, but is curved in such a manner that it has a slightly concave upper edge and a slightly convex lower edge. The technical advantage of this is that the eyelets have a position which allows secure hooking of the hook despite the non-flat body shape of the person. In a typical belt routing of a tightened tension band, in particular for a brace or orthosis, the lowest point of the belt is usually in the front, on the abdomen—that is, in the location where the coupling element is positioned. This means that the belt elements, seen from the coupling element, often do not run completely horizontally to the back. Rather, they run approximately horizontally and slightly upward. For this reason, the vertical position of the hook of the second coupling means is also slightly different, depending on where the hook is located in the abdominal area. However, the ladder-like eyelet element which is curved slightly upward enables the hook to catch effectively in each of the various eyelets, since the eyelets' position is higher as a function of how far they are from the abdominal center—that is, the lowest point of the belt route.

In a preferred embodiment, the eyelet element is designed like a ladder and has a plurality of eyelets, and is curved inward as seen from the associated first grip element. The advantage of such a curvature is that the ladder-like eyelet element is essentially adapted to the abdominal curve over the entire length of the eyelet element, and therefore does not protrude, accordingly increasing the wearing comfort. This is especially true if the eyelet element is formed by a relatively rigid or mostly inflexible material.

In a preferred embodiment, the eyelet element is therefore designed in the manner of a ladder and has a plurality of eyelets and, as viewed from the associated first grip element, is curved both upwardly and inwardly. This two-fold curvature leads to particularly good wearing comfort with simultaneously secure coupling of the coupling means.

In a preferred embodiment, the handle of the first and/or second grip element is positioned approximately horizontally. In a preferred embodiment, the handle of the first and the second grip element is positioned approximately horizontally. This applies in particular to a coupling element for an abdominal belt. Preferably, a handle is connected to the grip element on both lateral ends of a grip element.

The approximately horizontal embodiment of the handle and/or the connection to the lateral ends of a grip element has the advantage that the handle can have a sufficient size—specifically, width—of the grip opening to allow easy reaching into the grip opening with one hand, and that the handle lies along the tensile/tensioning forces in this position, such that it is particularly effective for tightening, in particular, an abdominal belt, wherein the grasping hand can have a favorable position and hold in this way.

In a preferred embodiment, the handle of the first and/or second grip element is designed such that it can be retracted. In a preferred embodiment, the handle of the first and second grip elements is designed such that it can be retracted. A retractable grip is advantageous because it can be easily flipped out for the tightening of the belt elements, and then can be easily retracted back in such that it does not interfere with the freedom of movement when the belt—for example, an abdominal belt of an orthosis—is worn. Preferably, the handle does not project forward out of the grip element when in the retracted position. In a preferred embodiment, the handle of the first and/or second grip element has a catch for the retracted position. The advantage of this is that the grip cannot be accidentally flipped out. In a preferred embodiment, the handle of the first and/or second grip element is configured such that it can be retracted in a downward direction.

In a preferred embodiment, the first coupling means is connected to the first belt element by means of a hinge, and/or the second coupling means is connected to the second belt element via a hinge. In a preferred embodiment, the first coupling means is connected to the first belt element via a hinge and the second coupling means is connected to the second belt element via a hinge. Preferably, the hinge runs approximately vertically in the case of an abdominal belt. Preferably, the hinge is positioned between one end of a belt element and a lateral end of a grip element. A hinge can advantageously increase wearing comfort, because the coupling means can buckle relative to the belt element connected via the hinge, without the belt element itself buckling.

In a preferred embodiment, the first belt element and/or the second belt element are components of a belt system and/or of a tensioning system via pulleys. In a preferred embodiment, the first belt element and the second belt element are components of a tensioning system via pulleys, in particular for braces and orthoses, and preferably back support orthoses. Such a tensioning system is disclosed, for example, in DE 10 2012 009 214 A1, the content of which is incorporated into the present application.

In a preferred embodiment, the first belt element and the second belt element form a belt of a brace or an orthosis. In a preferred embodiment, the first belt element and the second belt element form an abdominal belt of a brace, or particularly preferably an orthosis. As described above, the coupling element according to the invention is particularly suitable for closing of a belt, in particular an abdominal belt of a brace or orthosis. Preferably, the orthosis is a back support orthosis. The brace is preferably a back brace.

The present invention thus also relates to the use of a coupling element according to the invention in a brace or in an orthosis, in particular for a tension belt, in particular in the abdominal area, of a back brace or back support orthosis.

The present invention also relates to a brace or orthosis comprising at least one coupling element according to the invention. The brace is preferably a back brace. Preferably, the orthosis is a back support orthosis.

Preferably, the brace or orthosis comprises the coupling element according to the invention, to couple a tension belt, in particular in the abdominal area.

Particularly preferred is a back support orthosis having a tension belt system, in particular a tension belt system via pulleys, wherein the tension belt system can be reversibly closed in the abdominal area by means of a coupling element according to the invention.

Preferred embodiments of the present invention are also apparent from the dependent claims.

The invention is described in greater detail with reference to the following figures, wherein the embodiments shown in the figures should not be construed as limiting the invention.

In the drawings:

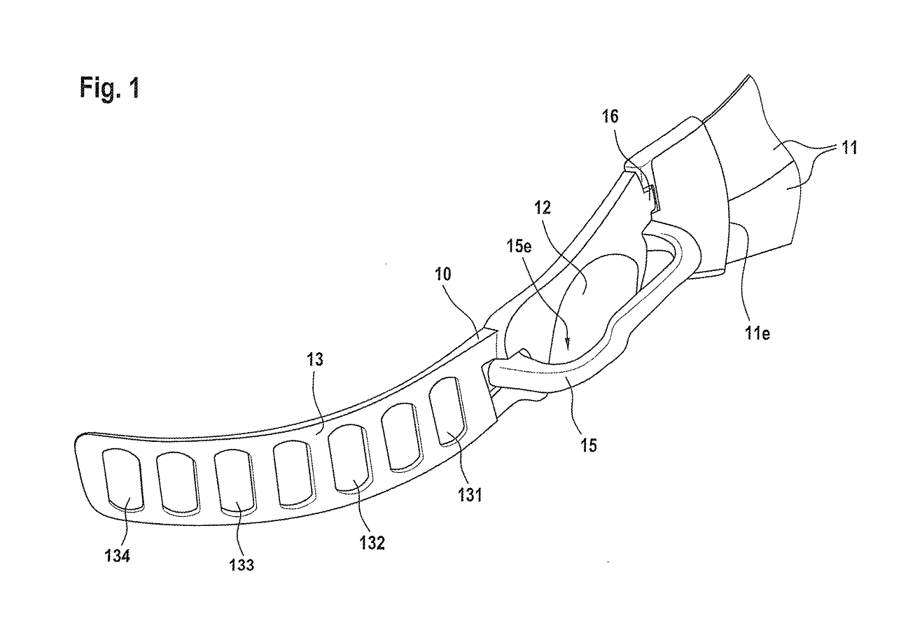

FIG. 1 shows a preferred embodiment of a first coupling means according to the invention, from the front,

FIG. 2 shows a preferred embodiment of a second coupling means according to the invention, from the front,

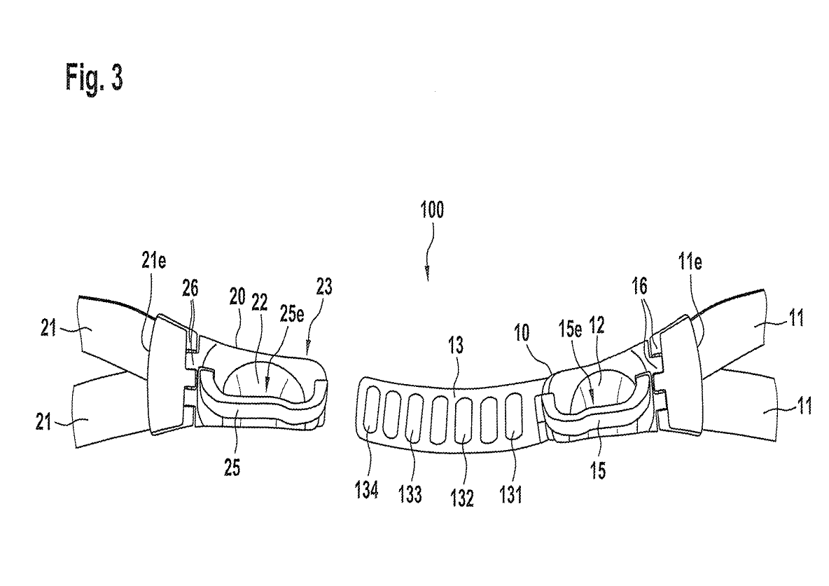

FIG. 3 shows a preferred embodiment of a coupling element according to the invention, with uncoupled coupling means, from the front,

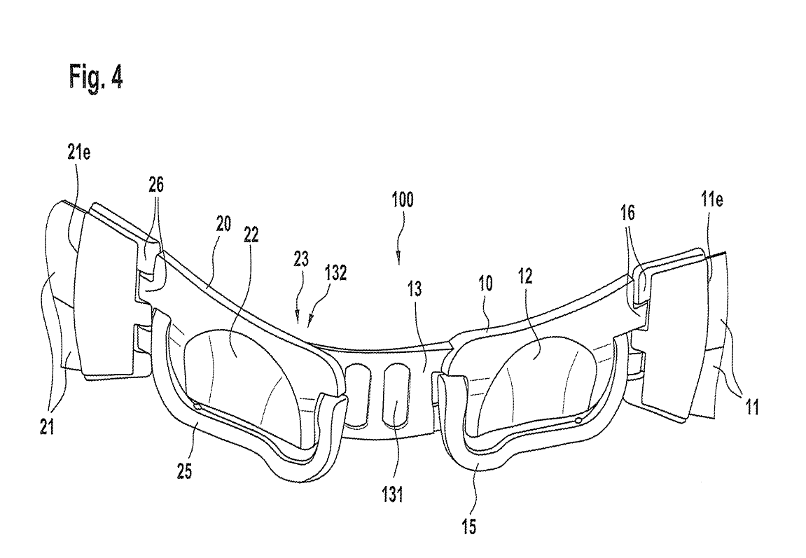

FIG. 4 shows the coupling element of FIG. 3, with coupled coupling means,

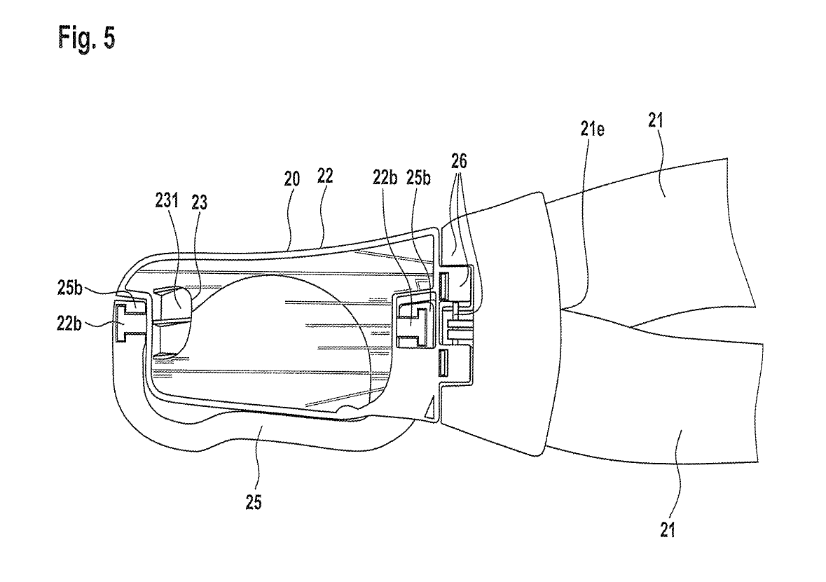

FIG. 5 shows the first coupling means of FIG. 2, from the back side,

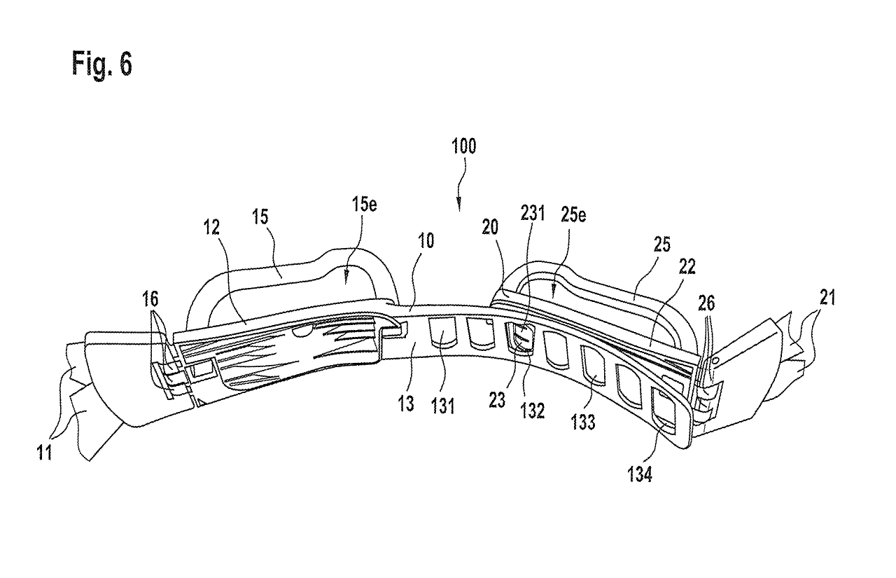

FIG. 6 shows the coupling element of FIG. 4, from the back side,

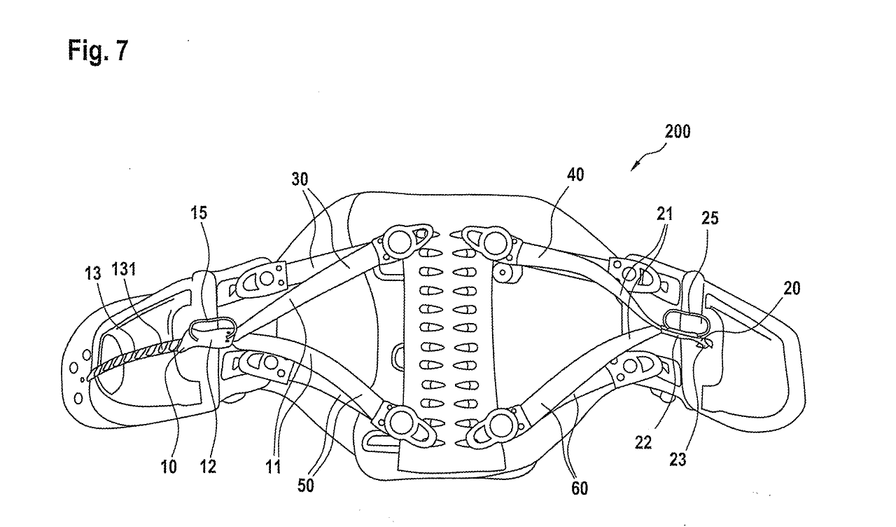

FIG. 7 shows a back support orthosis according to the invention,

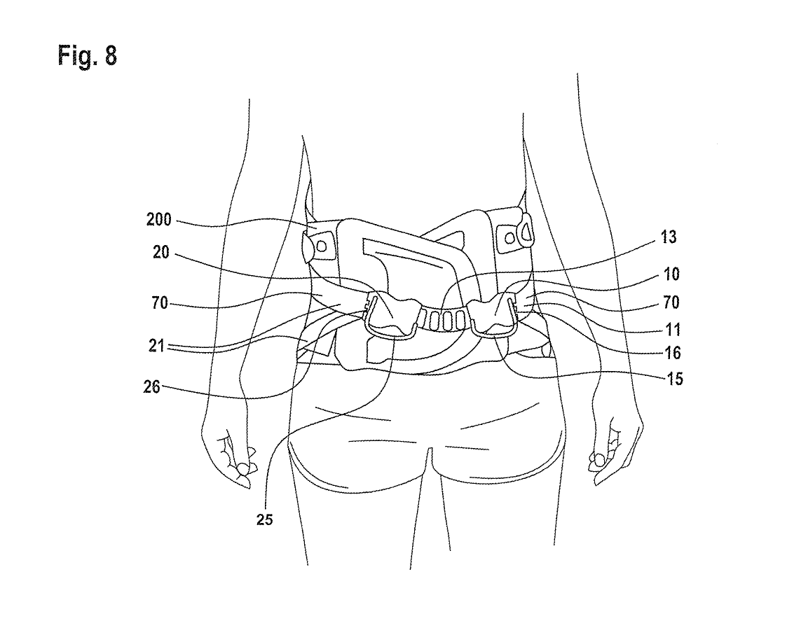

FIG. 8 shows the back support orthosis of FIG. 7 when worn.

FIG. 1 shows the front side of a preferred embodiment of a first coupling means (10) as a part of a coupling element according to the invention. The coupling means (10) is connected to a first belt element at the end (11e) of the belt element (11) via a hinge (16). The coupling means (10) has a grip element (12) and an eyelet element (13), wherein the grip element (12) is positioned between the eyelet element (13) and the belt element (11). The eyelet element (13) is designed in the form of a ladder, wherein the rungs of the ladder shape form a total of 7 eyelets (131, 132, 133, 134). In addition, the eyelet element (13) is curved both upward and inward as seen from the grip element (12). The grip element (12) has a grip (15) which is designed to be able to retract, and which is connected to the grip element (12) on the lateral ends thereof. A grip opening (15e) is formed as a result, and is wide enough so that a hand can grip the handle—in particular, four fingers of one hand can reach into the grip opening. The handle (15) is shown in the flipped-out position, and can be retracted in the downward direction.

FIG. 2 shows the front side of a preferred embodiment of the second coupling means (20) of a coupling element according to the invention, wherein the coupling means (20) is attached via a hinge (26) to one end (21e) of a second belt element (21). The coupling means (20) has a second grip element (22), wherein a handle (25) which can be retracted in the downward direction is attached on the sides thereof. The handle is shown in the retracted position in which it does not protrude forward, and thus does not create an interference. When the handle (25) is flipped out, it forms a grip opening (25e) for holding the handle (25). A hook element (23) with a hook is situated on the non-visible rear side of the grip element (22).

FIG. 3 shows the front side of a preferred embodiment of a coupling element according to the invention (100), having the coupling means shown in FIGS. 1 and 2 (10, 20). Accordingly, the first belt element (11) and the second belt element (21) can be seen here as well, each with its ends (11e, 21e) connected via hinges (16, 26) to the respective coupling means (10, 20). The first coupling means (10) has a first grip element (12) and an eyelet element (13) in this case as well. A handle (15) is hingedly connected to the sides of the grip (12). The eyelet element (13) again has a plurality of adjacent eyelets (131, 132, 133, 134). On the back side, the second coupling means (20) comprises the hook element (23) with a hook (not visible here). On the front side, the second handle (25) can be seen in the flipped-out position. In the flipped-out position, the two handles (15, 25) each form a grip opening (15e, 25e), wherein a hand can reach into each of the same.

The two coupling means (10, 20) of the coupling element (100) are shown here in the uncoupled position. To couple the two coupling means (10, 20) via one of the eyelets (131, 132, 133, 134) and the hook, both of the same are pulled toward each other in the usual manner. As a result, the belt elements (11, 21) become taut, such that a corresponding amount of force is required. With the handle (15, 25) included according to the invention, the grip elements (12, 22) can be easily gripped, such that the coupling means (10, 20) can be moved toward each other even by persons with limited mobility or low strength reserves in spite of the tension of the belt elements (11, 21). This is advantageous both during coupling and decoupling.

FIG. 4 shows the coupling element (100) of FIG. 3 once more in a front view—this time in a coupled/closed position. The two belt elements (11, 21) with their ends (11e, 21e), the adjacent hinges (16, 26), and the two coupling means (10, 20) with the corresponding grip elements (12, 22) can be seen here again. The handles (15, 25) are flipped down in this case, such that they do not create interference, and lie flat against the grip elements (12, 22). The hook element (23) (not visible) is hooked by its hook into one of the eyes (132) of the eyelet element (13). If the tensile force of the belt elements (11, 21) should be increased, or the person wearing the coupling element (100) has a smaller girth, the hook can advantageously be hooked into an eyelet (131) positioned closer to the grip element (12). The same applies, if lower tensile force is desired, or if the person has a larger girth, for eyelets which are positioned further from the grip element (12).

FIG. 5 shows the back side of the second coupling means (20) of FIG. 2, wherein the coupling means (20) is attached on one end (21e) of the second belt element (21) via a hinge (26). The coupling means (20) has a second grip element (22), wherein a handle (25) which can be retracted in the downward direction is attached on the sides thereof. The handle is shown once more in the retracted position. The handle (25) is movably connected via its ends (25b) to lateral elements (22b) of the grip element (22). Such a connection is preferably also included for the first handle of the first coupling means. A hook element (23) with a hook (231) which can be hooked into an eyelet of the first coupling means is situated on the back of the grip element (22).

FIG. 6 shows the coupling element (100) of FIG. 4 in a rear view—again in a coupled/closed position. The two belt elements (11, 21), the adjacent hinges (16, 26), and the two coupling means (10, 20) with the corresponding grip elements (12, 22) can be seen here again. The handles (15, 25) are flipped out, such that it is easy to reach into the grip openings (15e, 25e). The hook element (23) is hooked by its hook (231) on the rear side of the grip element (22) in one of the eyelets (132) of the eyelet element (13). Again, the hook (231) can alternatively be hooked into one of the other eyelets (131, 133, 134). The second coupling means (20) is sharply bent via the corresponding hinge (26) with respect to the second belt element (21). This illustrates the advantageous additional mobility resulting from such a hinge connection.

FIG. 7 shows a back support orthosis (200) according to the invention, which, for the purpose of closing a pulley tensioning system (30, 40, 50, 60) with the two belt elements (11, 21) in the abdominal area, has a coupling element according to the invention with the two coupling means (10, 20), with the grip elements (12, 22), with the eyelet element (13) with eyelets (131), and with the hook element (23) with hooks. Such a back support orthosis is known, for example, from DE 10 2012 009 214 A1, shown particularly in FIG. 2. In addition, the coupling means (10, 20) of the back support orthosis each have a handle (15, 25). These handles (15, 25) make it possible, in an advantageous manner, for the grip elements (12, 22) to be easily gripped as described above, and then for the coupling means (10, 20) to be coupled and uncoupled again in an improved manner, in spite of the tension of the belt elements (11, 21).

FIG. 8 shows the back support orthosis (200) of FIG. 7 when it is placed on the body of a person. The abdominal belt (70) formed by the belt elements (11, 21) is closed by the coupling means (10, 20) as described above. The handles (15, 25) are flipped down so that they do not create interference. However, they can be flipped out once more to remove the back support orthosis (200). The coupling means (10, 20) are again connected via hinges (16, 26) to the belt elements (11, 21). The hinges can advantageously increase wearing comfort, since the coupling means (10, 20) can therefore bend relative to the belt elements (11, 21) without the belt elements (11, 21) themselves being bent. FIG. 8 makes clear the other described advantages of the individually-shown elements, such as the good fit of the eyelet element (13) which curves upward and inward.

1-9. (canceled)

10. A coupling element for coupling a first belt element to a second belt element, in combination with the first and second belt elements, the coupling element comprising:
a first coupling means at a first end of the first belt element;
a second coupling means at a first end of the second belt element,
wherein the first coupling means has a first grip element and an eyelet element with at least one eyelet, and wherein the second coupling means has a second grip element and a hook element with a hook to hook into the eyelet of the eyelet element, the first grip element and the second grip element have a handle with a grip opening for the holding of the first and/or second grip element, wherein the handle of the first and second grip elements is retractable.

11. The coupling element according to claim 10, wherein the first belt element and the second belt element form an abdominal belt.

12. The coupling element according to claim 10, wherein the first belt element and the second belt element form a tension band.

13. The coupling element according to claim 10, wherein the first belt element and the second belt element form a tension belt.

14. The coupling element according to claim 10, wherein the eyelet element is designed like a ladder, and has a plurality of eyelets, and, as viewed from an associated first grip element, is curved both upward and inward.

15. The coupling element according to claim 10, wherein the handle of the first and/or second grip element is positioned horizontally.

16. The coupling element according to claim 10, wherein the first coupling means is connected to the first belt element by means of a hinge, and/or the second coupling means is connected to the second belt element via a hinge.

17. The coupling element according to claim 10, wherein the first belt element and/or the second belt element are parts of a belt system and/or a tensioning system via pulleys.

18. The coupling element according to claim 10, wherein the first belt element and the second belt element form a belt, in particular an abdominal belt, of a brace or an orthosis.

19. The coupling element of claim 10 in combination with a brace or orthosis.
